(19) United States
(12) Patent Application Publication
(10) Pub. No.: US 2023/0226087 A1
(43) Pub. Date: Jul. 20, 2023

(54) PHOSPHOLIPIDS AND PHOSPHOLIPID METABOLITES FOR TREATING VIRAL AND BACTERIAL PNEUMONIA AND SEPSIS

(71) Applicant: ANDREAS HETTICH GMBH & CO. KG, TUTTLINGEN (DE)

(72) Inventor: ULRICH MASSING, MERZHAUSEN (DE)

(21) Appl. No.: 17/996,370

(22) PCT Filed: Apr. 16, 2021
(86) PCT No.: PCT/EP2021/059881

§ 371 Date: Oct. 17, 2022

(30) Foreign Application Priority Data
Apr. 17, 2020 (EP) 20170199.2

(51) Int. Cl. A61K 31/685; A61K 9/00; A61K 45/06

(52) U.S. Cl. CPC A61K 31/685; A61K 9/0053; A61K 45/06

(57) ABSTRACT

The invention relates to lyso-phosphatidylcholine (LysoPC), or a suitable precursor or derivative thereof, or a composition containing LysoPC and/or one or more suitable precursors or derivatives thereof, for use in the treatment and aftercare of inflammatory diseases in humans that involve lowering of the LysoPC level, including the treatment, prevention or support of treatment and aftercare of viral and bacterial pneumonias and sepsis, including pneumonia and sepsis as a consequence of influenza, Covid-19, ARDS, cancer, for supporting immunotherapy in cancer in view of effectiveness and for reducing side-effects, such as pneumonitis, colitis or hepatitis, and for reducing undesirable vaccination responses. The invention further relates to alpha-glycerophosphocholine (alpha-GPC), or a variant thereof, or a composition containing alpha-GPC and/or one or more variants thereof, for use in the treatment and aftercare of cancers and tumor cachexia.

The invention relates to lyso-phosphatidylcholine (LysoPC), or a suitable precursor or derivative thereof, or a composition containing LysoPC and/or one or more suitable precursors or derivatives thereof, for use in the treatment and aftercare of inflammatory diseases in humans that involve lowering of the LysoPC level, including the treatment, prevention or support of treatment and aftercare of viral and bacterial pneumonias and sepsis, including pneumonia and sepsis as a result of influenza, Covid-19, ARDS, cancer, for supporting immunotherapy in cancer in view of effectiveness and for reducing side-effects, such as pneumonitis, colitis or hepatitis, and for reducing adverse vaccination responses. The invention further relates to alpha-glycerophosphocholine (alpha-GPC), or a variant thereof, or a composition containing alpha-GPC and/or one or more variants thereof, for use in the treatment and aftercare of cancers and tumor cachexia.

### BACKGROUND OF THE INVENTION

Pneumonias are severe diseases, which may have a lethal outcome especially in persons of higher age and with underlying medical conditions. Pneumonias may be of unknown origin, but are often caused by viruses as well as bacteria. A bacterial pneumonia often follows a viral one. The inflammation in the pulmonal tissue is caused, among other things, by the immune response against viral or bacterial pathogens in the pulmonal tissue, which is often very heavy. The inflammatory reaction in the pulmonal tissue may cause water to intrude into the alveoli, which renders gas exchange more difficult. In addition, the amount of surfactant lipids (especially of phosphatidylcholine) in the alveoli is reduced, and accordingly the surface tension increases significantly (up to 35 mN/m in pneumonias and ARDS), which additionally complicates the task of keeping the alveoli open and consequently impedes the breathing work (Gunther, A. et al., Am. J. Respir. Crit. Care Med., 153(1): 176-184 (1996)).

The response of the immune system may become very heavy in a pneumonia, so that a so-called cytokine storm arises. In this case, the very heavy inflammatory reaction no longer proceeds in a controlled way, and its clinical course is similar to that of a sepsis, which means that the immune response is no longer primarily directed to the inflamed organ and the cause of the inflammation, e.g., viruses, whereby different other organs and tissues may be damaged, in part heavily.

Against various pneumonia-causing viruses or bacteria (e.g., pneumococci or influenza viruses), there are vaccinations, which may have side-effects in singular cases, such as, for example, vaccination against influenza. A strong response of the immune system to the vaccination may lead to influenza-like symptoms, and symptoms such as shivering, fatigue, or muscle pain are seen.

Also in a sepsis, an immune response proceeds that is no longer controlled, which is induced, inter alia, by bacterial toxins (especially bacterial lipopolysaccharide (LPS)), and as a result thereof, organ failure may arise, including of the lung, although the inflammatory reaction is not focused on this organ at all. A sepsis or septic shock may also be the result of cancer, polytrauma, or severe inflammations (e.g., pneumonia after COVID-19 or influenza).

The “Acute Respiratory Distress Syndrome (ARDS)” is a reaction or inflammation of the lung as a response to damaging factors, irrespective of whether the pulmonal inflammation is primarily induced pulmonarily or systemically. As in a pneumonia, inflow of water into the lung and a change or reduction of the lung surfactant may occur, whereby gas exchange is disturbed (Gunther, A. et al., Am. J. Respir. Crit. Care Med., 153(1): 176-184 (1996)).

More and more often, cancers are treated by immune therapies. In such immune therapies, the immune system is enabled to trigger an immune response against cancer cells, for example, by using checkpoint inhibitors. Unfortunately, this promising therapy (e.g., with ipilimumab, or nivolumab, or combinations of the two inhibitors) has its limitations. Thus, on the one hand, an immune response, i.e., to the therapy, is seen only in part of the cancer patients. When the immune therapy fails, there may be an immune deficiency, perhaps caused by the cancer, i.e., the immune system cannot be activated as expected. On the other hand, the immune reaction may also get out of control, and an important example thereof is the occurrence of pneumonitis, colitis or hepatitis, all of which may be life-threatening (Kähler, KC., Pharmakon, 6: 463-468 (2018)). A pneumonitis is an inflammatory alteration of the lung, which, in contrast to pneumonia, is not caused by microorganisms (bacteria, fungi) or viruses, but by lung-damaging impacts, wherein ionizing radiation, drugs or, e.g., chemical or physical noxes are important. In order to avoid damage to organs upon the occurrence of the above-mentioned side-effects, the triggered immune response must be stopped, e.g., with corticoids, such as methylprednisolone.

Phospholipids (hereinafter briefly referred to as “PL”) are a main component of animal cell membranes. They usually consist of a hydrophilic head, which is linked to hydrophobic non-polar residues through a negatively charged phosphate group. The PLs most frequently found in biological membranes are glycerophospholipids.

Glycerophospholipids (hereinafter briefly referred to as “GPL”) have a structure as shown in formula (I):

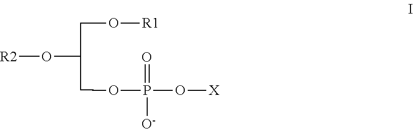

They consist of a hydrophilic head, which is linked in sn-3 position to a glycerol skeleton and additionally to one or two hydrophobic non-polar radicals R¹ and R² through a negatively charged phosphate group (IUPAC Compendium of Chemical Technology, 2^{nd} Ed. (1997)). The latter are usually O-acyl radicals from fatty acids having a length of 14-24 carbon atoms, but may also be O-alkyl or O-1-alkenyl radicals. GPLs with one or two O-acyl radicals are also referred to as 1-acyl-, 2-acyl- or 1,2-diacylglycerophospholipids, or generically summarized as acylglycero-phospholipids (hereinafter briefly referred to as “AGPL”). Typical AGPLs include phosphatidylglycerol, phosphatidylserine, phosphatidylethanolamine (hereinafter referred to as “PE”), phosphatidylinositol (hereinafter referred to as “PI”), phosphatidic acid (hereinafter referred to as “PA”), and phosphatidylcholine (hereinafter referred to as “PC”). Acylglycerophospholipids are contained in lecithin, in particular, and their main component is mostly PC.

Glycerophospholipids, especially PCs or PC-containing mixtures, such as lecithin, find use as a component of food supplements and foodstuffs, in cosmetics, in parenteral nutrition, and as auxiliaries for the formulation of medicaments, because of their emulsifying effect. Non-hydrogenated phospholipids from soybean or egg are mostly used, and hydrogenated phospholipids are preferred only for the formulation of medicaments for intravenous application.

Glycerophospholipids are also known as active components of food supplements and medicaments (e.g., marine phospholipids (from salmon roe or krill)), which contain a high proportion of long-chained ω-3-fatty acids, and are known, e.g., for supporting the therapy of prostate cancer, or for the treatment of cachexia (EP-A-1745788). Further examples include soy phospholipids, e.g., contained in Essentiale® for the therapy of liver diseases, in Buer-Lecithin plus Vitamine® for the therapy of states of exhaustion, and for strengthening the nerves, or in Lipostabil® for the therapy of increased lipid levels, wherein the recommended doses range from 1.5 g to about 6.5 g of PL/day (all the data are from the package inserts).

The absorption of glycerophosphocholines in the gastrointestinal tract upon oral uptake thereof does not occur directly. PC is cleaved at first by the pancreatic phospholipase A2 into LysoPC and free fatty acids, which are both taken up by mucosal cells in the intestine (enterocytes). The opinion currently prevailing in the literature is that the major part of LysoPC is hydrolyzed further in the mucosal cells, about ⅓ is reacylated to PC according to the current state of knowledge, and subsequently become a component of chylomicrons, which are released into the lymphatic system (Parthasarathy, S. et al., Biochem. J., 140(3): 503-508 (1974)).

Further, hydrogenated PC is known for reducing tumor metastases (as a liposome for i.v. application). An effect thereof on the immune system has not been described.

Phospholipids are further used for the formulation of compositions for parenteral nutrition. Such formulations include emulsions, typically consisting of 10, 20 or 30% of triglycerides (e.g., soybean oil, MCT (medium-chain triglycerides), olive oil, fish oil, or mixtures thereof (e.g., soybean/olive 4:1)). The emulsions further contain a minor proportion of phospholipids (lecithin), e.g., from hen's eggs, as emulsifiers, wherein as low a phospholipid-to-triglyceride ratio as possible is sought (e.g., Intralipid, Baxter, 10% (20%; 30%) emulsion: 10 g (20 g; 30 g) of soybean oil, and 0.6 g (1.2 g; 1.2 g) of phospholipid/100 ml; Lipundin 10% N, Braun: 8 g of lecithin per 100 g of soybean oil; Lipofundin MCT 10%, Braun: 8 g of lecithin per 100 g of soybean oil/MCT (1:1)). Emulsions having a higher content of triglycerides evidently require less phospholipid for emulsification, which is considered as advantageous. For a low phospholipid-to-triglyceride ratio, a better metabolic tolerability is found with a significantly lower accumulation of phospholipids and cholesterol in the plasma (Hartig, W. et al. (Eds.), Ernährungs-und Infusionstherapie, 8th Edition, Thieme (2004)). The lecithins employed are highly enriched lecithins with a glycerophosphatidylcholine proportion of typically 75% or 80% (e.g., Lipoid E 75/E 80).

Alpha-glycerophosphocholine (alpha-GPC) consists of a glycerol basic unit to which a phosphocholine group is bound at its sn-3 position as a phosphodiester. Alpha-GPC is formally derived from phosphatidylcholine, in which the two fatty acid radicals have been removed by hydrolysis.

Alpha-GPC is employed as an active ingredient in food supplements, serving as a choline source. Alpha-GPC has a positive effect on the gain of physical strength from workout (Bellar, D. et al., J. Int. Soc. Sports Nutr. 12: 42 (2015); Marcus, L. et al., J. Int. Soc. Sports Nutr. 14: 39 (2017)), and shows positive effects in patients with Alzheimer's disease and other dementia phenomena (Review: Parnetti, L. et al., Mech. Ageing Dev. 122(16) 2041-2055 (2001)), and improves the cognitive performance (Suchy, J. et al., Nutr. Res. 29(1) 70-74 (2009)), and awareness (Hoffman, JR. et al., J. Int. Soc. Sports Nutr. 7: 39 (2010)). It is discussed whether alpha-GPC slows down the process of ageing (Matsubara, K. et al., Biosci. Biotech. Biochem. 82(4) 647-653 (2017)). Typical doses are around 500 to 1,200 mg/day. An effect on the immune system or an effect on cancers including attendant symptoms thereof, such as cachexia or fatigue, has not been known.

In yeasts (Saccharomyces cerevisiae), alpha-GPC additionally serves directly as a precursor in the biosynthesis of LysoPC. In this case, a GPC-acyltransferase (GPCAT or Gpc1) that acylates alpha-GPC in an acyl-CoA-dependent way has been described (Stalberg, K. et al., J. Lipid Res. 49: 1794-1806 (2008); Anaokar, S. et al., J. Biol. Chem. 25; 294(4): 1189-1201 (2018)). The new enzyme from yeast has already been cloned and shows little relationship with other acyltransferases from the lipid field (Glab, B. et al., J. Biol. Chem. 291(48): 25066-25076 (2016)). The activity of this enzyme was also detected in safflower. GPCAT activities in animal or human tissues have not yet been detected.

1- and 2-acylglycerophospholipids are also referred to as lyso-phospholipids (LysoPL). Lyso-Phospholipids are single-chain phospholipids formed from 1,2-diacylphospholipids by phospholipase-A-catalyzed cleavage or by the chemical hydrolysis of a fatty acid radical. LysoPL have surface-active properties enabling them to lyse red blood cells (hence the name). For this reason, hemolytic properties were adopted upon application of the pure lyso-phospholipids in relevant doses, as also shown by different phospholipids employed for cancer control that are not acylglycerophospholipids. (U.S. Pat. Nos. 4,562,005; 4,775,758; EP 0171968; U.S. Pat. Nos. 5,489,580; 6,172,050; WO 01/72289; DE 4408011; Zeisig, R. et al., Anticancer Drug Des. 16(1): 19-26 (2001); Arndt, D. et al., Breast Cancer Res. Treat. 43(3): 237-46 (1997); DE-A-3304870; U.S. Pat. No. 4,492,659; DE-A-3204735; U.S. Pat. No. 4,565,659; Modolell, M. et al., Cancer Res. 39(11): 4681-4686 (1979)).

Lyso-phospholipids (1-acyl- or 2-acylglycerophospholipids) may be formed from the different two-chained membrane-forming phospholipids. Examples of lyso-phospholipids include lyso-phosphatidylcholine, lyso-phosphatidylethanolamine (lyso-cephalin), lyso-phosphatidylglycerol, -serine, and lyso-phosphatidic acid, all of whose starting phospholipids are 1,2-diacylglycerophospholipids. Lyso-phospholipids have also been known as cancerostatic compounds (U.S. Pat. No. 4,372,949).

In contrast to double-chained phospholipids, lyso-phospholipids are not currently employed as therapeutic agents. However, it is known that any formulation with a double-chained phospholipid always also contains minor proportions of its corresponding natural degradation product, namely the corresponding lyso-phospholipid.

LysoPC is a central metabolic product in the human body. The current state of knowledge for the generation of LysoPC in the human body is that LysoPC is formed in the cleavage of a fatty acid from the frequently occurring phosphatidylcholine (lecithin). PC is in turn provided by the reacylation of LysoPC or via the Kennedy pathway, or the phosphatidylethanolamine pathway (Lands, WEM. et al., J. Biol. Chem. 231(2): 883-888 (1958); Kennedy, EP. et al., J. Biol. Chem., 222(1): 193-214 (1956); Jacobs, RL. et al., J. Biol. Chem. 285(29): 22403-22413 (2010)). Other biosynthetic pathways for LysoPC or PC in humans have not been described.

Because of the distribution of saturated and unsaturated fatty acids in phospholipids (sn1 position: predominantly saturated fatty acids, sn2 position: predominantly unsaturated fatty acids), predominantly saturated LysoPC should be formed when LysoPC is formed from phospholipids by cleaving off a (predominantly unsaturated) fatty acid from the sn-2 position of a PC by phospholipase A2. However, surprisingly, only a very slight excess of saturated LysoPC species is found in the plasma. (e.g., Zhao, Z. et al., J. Clin. Oncol. 25(19) 2696 ff. (2007: 162 μM of saturated LysoPC (55%) vs. 129 μM of unsaturated LysoPC (45%)).

As can be seen from the following discussion, research work about LysoPC in humans have focused on the concentration of this molecule in the plasma or serum, where it is predominantly in a form bound to albumin. Albumin has 3-4 binding sites for LysoPC and binds about 80% of the plasma LysoPCs (Switzer, S. et al., J. Lipid Res. 6: 506-511 (1965)).

The mean level of LysoPC in the plasma of humans was examined in different studies, of which some examples include: 280 μM (Drobnik, W. et al., J. Lipid Res. 44: 754-761 (2003); 292±74 μM (Zhao, Z. et al., J. Clin. Oncol. 25(19) 2696 ff (2007); 234 μM (Kishimoto, T., et al., Clin. Biochem. 35: 411-416 (2002). Some authors found slightly lower values for healthy women as compared to men (365 vs. 340 μM: Süllentrop, F. et al., NMR Biomed. 15(1) 60-68 (2002); 270 vs. 230 μM: Gillett, MPT. et al., Atherosclerosis 22: 111-124 (1975). The LysoPC values in the respectively examined collective of healthy subjects always had a considerable variation width, which indicates individually different “optimum LysoPC values” in different humans. Nevertheless, it seems to be advantageous for a human to tend to have rather high LysoPC values. Thus, it has been found that high levels of LysoPC species that include stearic acid as the fatty acid lead to a lower risk for breast cancer, prostate cancer, and for colorectal tumors (Kühn, T. et al., BMC Medicine 14: 13 (2016). In addition, it has been shown that the serum LysoPC levels are significantly increased in humans having reached an old age (Montoliu, I. et al., Aging 6(1) 9-25 (2014)—supplementary tables).

From the above mentioned studies, an approximate average LysoPC plasma level in healthy persons of about 300 μM is obtained, which means, for example, for a man having a plasma volume of about 3.5 liters, that about 500 mg of LysoPC are in the plasma. The LysoPC half life in humans has never been determined, but is probably in the range of a few hours. In squirrel monkeys, the half life was determined to be 1.3 hours (Portman, OW. et al. J. Lipid Res. 11:596-604 (1970)), and about 4 hours in mice (doctoral thesis by Anna Raynor, Uni-Freiburg, 2015). For an estimated value for humans of 2.5 hours, a conversion rate of about 2-3 g of LysoPC/day would be obtained.

The generation of LysoPC in humans is not comprehensively known. It is known that LysoPC is formed, inter alia, by the hydrolysis of phospholipids in lipoproteins, e.g., by the enzymes LCAT (lecithin-cholesterol acyltransferase), or by endothelial lipase. Assuming that the complete PL envelope of chylomicrons (containing a maximum of 10% PL) was hydrolyzed to LysoPL, this would correspond to a fat absorption of about 50 g of fat (triglycerides), about 5 g of PL, which would correspond to about 3-4 g of LysoPL. A maximum of 6-12 g of LysoPC could be generated from the hydrolysis of phosphatidylcholine from bile, if the 10-20 g of PC (Northfield, TC., et al. Gut, 16: 1-17 (1975)) that arrive at the GI tract daily with the bile were hydrolyzed to LysoPC and then also absorbed completely, which is not the case (see below). Additionally, there would be a few milligrams to a few grams of LysoPC from the cleavage of phospholipids from the food in the GI tract. Thus, an egg contains about 1 g of PC. However, as described above, a major portion of the LysoPC in the mucosal cell is hydrolyzed further, and only about ⅓ is reacylated to PC and subsequently becomes a part of chylomicrons, which are released into the lymphatic system (Parthasarathy, S. et al., Biochem. J., 140(3): 503-508 (1974)). A direct release of LysoPC into the blood or lymphatic system has not been described.

Also, the consumption of LysoPC in the body has not yet been examined comprehensively, but some processes are known or probable. Thus, it has already been shown that the supply of the central nervous system with long-chained and highly unsaturated fatty acids and (probably) choline is effected through the specific uptake of long-chained and highly unsaturated LysoPC species (Bernoud, N. et al., J. Neurochem. 72: 338-345 (1999)). Recently, a sodium-dependent LysoPC transporter of the central nervous system has been found and characterized (MFSD2A), which seems to be the first member of a superfamily of LysoPC transporters (Nguyen, LN. et al. Nature 509: 503-506 (2014)).

Pulmonary surfactant is prepared by alveolar macrophages type II and predominantly consists of saturated phosphatidylcholine and the surfactant proteins A to D (Agassandian, M. et al., BBA 1831(3): 612-625 (2013). Pulmonary surfactant spreads over the humid surface of the very small alveoli and represents the boundary towards the air. The pulmonary surfactant significantly reduces the surface tension of alveolar water and mainly contributes to ensuring that the alveoli will not collapse from too high a surface tension, and that the breathing work (for inflating the alveoli) remains low.

Pulmonary surfactant largely consists of phosphatidylcholine (PC) and is constantly renewed by alveolar macrophages type II for maintaining the gas exchange in the alveoli (Goss, V. et al. BBA 1831: 448-458 (2013)). The necessary PC is prepared by simple reacylation from LysoPC, and the key enzyme in the biosynthesis of saturated PC species is LPCAT 1 (lyso-phosphatidylcholine acyltransferase 1), which converts LysoPC to a PC with consumption of acyl-CoA (Agassandian, M. et al., BBA 1831(3): 612-625 (2013)). It is apparent that both the LysoPC from the alveolar macrophages themselves and from the periphery will play a role for the synthesis of pulmonary surfactant. In experiments with squirrel monkeys, it has been found that a large amount of the radioactivity accumulates in the lung upon delivery of radioactively labeled LysoPC (Portman, OW. et al. J. Lipid Res. 11: 596-604 (1970)), In addition, it is conceivable that the protection of the lung from infections, which is also controlled through alveolar macrophages type II, will decrease for low LysoPC levels (i.e., the same cells that produce the pulmonary surfactant also process and present the pathogens and induce the immune response in the alveoli (American Journal of Respiratory & Critical Care Medicine, 2008: 179(5), pp 344-55)).

In squirrel monkeys, a very high uptake of radioactivity has been found in the kidneys upon delivery of radioactively labeled LysoPC (Portman, OW. et al. J. Lipid Res. 11: 596-604 (1970)). To what purpose LysoPC is required in the kidney is not known.

In addition, LysoPC seems to be required in a larger amount, in particular, in pathological processes, because reduced LysoPC levels are observed in many diseases and in special circumstances of life. Examples thereof include humans with cancers, patients with heavy immune reactions (sepsis), people with diabetes (type 2), and overweight people (Barber, Minn. et al. Plos one 7(7): e41456 (2012)), or elderly people (Johnson, AA. et al., Aging Cell, 18(6): e13048 (2019)).

In various scientific publications, it has been shown that LysoPC levels are almost constantly reduced in cancer patients, especially in advanced cancers. Thus, for example, Zhao et al. found levels of 228 μM in CRC patients (controls: 292 μM) (J Clin Oncol 2007, 25(19): 2696), and Taylor et al. found 207 μM in various tumor entities (Lipids Health Dis. 2007, 6: 7). The variances of the values are higher in cancer patients as compared to the healthy controls. The LysoPC levels are further reduced when the patients are not able to feed themselves adequately, e.g., because of disturbed appetite, cachexia, etc. (Lipids Health Dis. 2007, 6: 7).

In the meantime, it has been found that one important reason for the reduced LysoPC levels in cancer patients is an increased LysoPC conversion, because solid tumor cells, in particular, very quickly degrade larger amounts of LysoPC, for utilization as energy source, inter alia (Lipids Health Dis. 2015, 14: 69). Thus, calculation based on cell culture data yields a LysoPC degradation of about 1 g of LysoPC per g of active tumor cells per day, and thus, correspondingly aggressive and/or large tumors may lead to a dramatic decrease of LysoPC while the overall conversion is increased.

EP 050365 described the therapeutic influence of phospholipids on tumor-induced cachexia, but a possible use of alpha-GPC is not mentioned therein. Tumor cachexia is a frequent syndrome in tumor patients with drastic weight loss and an altered body composition. There is degradation of the adipose tissue, of the skeletal muscles, and adverse affection of immunological defense mechanisms (Tisdale, M. J., Nutrition 17(5): 438-442 (2001); Tisdale, M. J., Curr. Opin. Clin. Nutr. Metab. Care 5(4): 401-405 (2002)). In the use of phospholipids for treating cachexia as described in EP 050365, the doses to be employed range from very low to very high amounts of phospholipids (2-300 mg/kg).

Fatigue after a cancer disease or cancer treatment, but also after other diseases, such as, e.g., COVID-19 (long COVID), is considered a multifactorial disease. In the case of cancer, both the cancer disease as such and the cancer treatment, e.g., by chemotherapy, irradiation and by selective therapies, seem to be able to initiate fatigue, since all these therapies also affect healthy cells. During or shortly after cancer therapy, up to 90 percent of the patients suffer from fatigue, which becomes chronic in an estimated 20 to 50 percent of the patients. Typical symptoms of fatigue in humans include reduced performance, a high need to sleep, sustained feeling of tiredness, a lack of motivation and drive, and, much like with a depression, subsiding interest, sadness, anxiety, disturbed focusing, increased distractibility, word-finding difficulty.

However, fatigue is also a known syndrome after surgery, or after severe inflammatory diseases, especially including COVID-19. In a fatigue rat model, it could be shown that the severeness of fatigue correlates with a reduction of LysoPC level (Y. Lu et al., J. Clin. Biochem. Nutr. 58: 210-215 (2016)).

One important conventional treatment approach for fatigue is movement therapy. Sports can prevent fatigue or reduce fatigue symptoms that are already manifest. The movement training aims at the preservation and build-up of fitness and muscles. However, a load that is significantly too heavy may in turn lead to deterioration of the fatigue symptoms.

LysoPC also plays an important role in immune responses. Thus, it has been found that the triggering of a strong systemic immune response (sepsis) results in a dramatic reduction of the LysoPC level. Thus, in a study with 100 sepsis patients, a mean LysoPC value of only 95 μM was measured (healthy controls in this study: 280 μM), wherein the risk of death was highest with the lowest LysoPC values (J. Lipid Res. 2003, 44: 754-761). LysoPC was proposed in combination with other parameters as a prognostic factor for survival in sepsis (Arshad, H., et al., J. Transl. Med. 2019, 17: 365, and Law, S.-H., et al., Int. J. Mol. Sci. 2019, 20: 1149).

Further, LysoPC administration is described in a peritoneal sepsis and pneumonia model in mice (Younes, S., et al., Antimicrobial Agents and Chemotherapy 2015, 59(7): 3920-24).

Not only sepsis seems to lead to increased turnover and thus to lower LysoPC levels. In a study, LysoPC was determined before and after a surgery of colorectal carcinomas. The LysoPC values are significantly decreased by the surgery and the accompanying immune response. Patients with post-surgery complications exhibited significantly lower LysoPC values as compared with patients with no complications (Surg Today 2018, 48(10): 936-943).

The expansion of immune cells and some further immune functions seem to be dependent on LysoPC, which was shown by the extremely high LysoPC reduction in sepsis patients (see above). In addition, it has been found in vitro that LysoPC supports the activation of the immune system and also induces INFγ and TNFα secretion. Macrophages are activated, and so are B cells (Huang, YH. et al., Clin. Exp. Immunol. 116: 326-331 (1999); Yamamoto, N. et al., J. Immunol. 147: 273-280 (1991). In vitro, it has been shown with NK cells that LysoPC induces the autophosphorylation of PKC_{ζ}, which results in a stimulation of NK cell activity (Toxicon 2007, 50(3): 400-410). LysoPC seems to be necessary in a double function as a signaling molecule and as a metabolite/building block for a quick immune cell activation and proliferation.

Consequently, a sufficient amount of LysoPC is very important to an adequate immune response and to surviving a sepsis, which was confirmed in different animal-experimental works.

Thus, it has been shown that LysoPC (s.c.) improves the survival of mice in a sepsis model based on E. coli very much in a dose-dependent way (from about 20% to 90% survival in 20 mg/kg LysoPC). In addition, it could be shown that a multiple administration over 48 hours and thus a LysoPC level that was permanently increased in this period increased best the long-term survival of the mice (a single injection increased the LysoPC level in the mice for only about 4 hours (Nature Medicine 2004, 10 (2): 161-167).

In a rat model of septic shock (Gram positive and Gram negative), it was shown that LysoPC (up to 10 mg/kg, i.v.) can reduce the damage to the organs and dysfunctions significantly, while higher doses of up to 100 mg/kg prove to be incapable or even harmful. The protecting effects of LysoPC were accompanied by a reduction of the IL1-β levels (Brit. J. Pharmacol 2006, 148: 769-777).

In two more sepsis models in mice, it was shown that the injection (i.p.) of 25 mg/kg LysoPC increased the survival of the animals from 0% to 69% and 40%, respectively (Antimicrob Agents Chemother 2015, 59: 3920-3924), and the bacterial clearance improved in the same way. The results were confirmed by the same group one year later, and in addition, it was found that the administration of different antibacterial substances synergistically improves the effect of LysoPC (Antimicrob Agents Chemother 2016, 60: 4464-4470),

In addition, LysoPC shows an activity as an adjuvant when administered simultaneously with various antigens in mice. The effect of LysoPC in the production of antigen-specific antibodies was identical to that of alum. LysoPC also induces cytotoxic cells (Vaccine 2006, 24(9): 1254-1263).

Although LysoPC plays a role in the activation of an immune response, increased amounts of LysoPC levels are evidently required after the activation for the immune response, which is manifested in the above mentioned reduced LysoPC levels. If critically low LysoPC levels are reached in a very strong immune response, including but not limited to sepsis, a vicious circle is obviously triggered. The (over)activated immune system “consumes” much LysoPC, but does not succeed in neutralizing the cause of the inflammation (e.g., a pathogen), which results in further lowering of the LysoPC level. The critically lowered LysoPC levels are then no longer sufficient for supplying the as such healthy organs, which may contribute to organ failure. In addition, it may be assumed that LysoPC also plays a regulating role in immune responses, which is why a strongly lowered level possibly even accelerates an exuberant immune response. Thus, it is known that LysoPC induces the differentiation of regulatory T cells, and stimulates the TGF-β production (Hasegawa, H. et al., Biochem. Biophys. Res. Commun. 415: 526-531 (2011)).

The relationships shown can also explain the fact that cancer patients often respond poorly to immune therapies, e.g., with checkpoint inhibitors, but also why immune therapies often must be stopped because of an overreaction of the immune system. The LysoPC level, which is lowered in an immune therapy in cancer patients because of an increased consumption by tumor cells, may on the one hand have the effect that the immune reaction cannot be activated correctly, or else that a strong immune reaction, which is desired in principle, leads to undesirable side effects, such as pneumonitis, colitis or hepatitis, because of the additional lowering of the LysoPC levels.

LysoPC plasma levels are also lowered in patients with overweight and/or diabetes type II (Barber, Minn. et al. Plos one 7(7): e41456 (2012)), which could exhaust the capacity of the immune system (see above) more quickly as compared to normal weight, non-diabetic patients in critical situations, e.g., in a sepsis. Also in elderly people, there is a trend towards lower LysoPC plasma levels (Johnson, AA. et al., Aging Cell, 18(6): e13048 (2019)).

In addition, it could be shown in a rat model that the fatigue problems, which often arise after inflammatory diseases or cancer or cancer treatment, correlates with a reduction of the LysoPC levels (Y. Lu et al., J. Clin. Biochem. Nutr. 58: 210-215 (2016)).

To conclude, it can be shown that many diseases, especially life-threatening ones, are associated with a decrease, in part strong decrease, of the LysoPC levels in the body. Examples include pneumonias, sepsis, influenza, COVID-19, ARDS, or pneumonitis, colitis, hepatitis after immune therapy in cancer. Such low levels could have the consequence that many organs depending on LysoPC can no longer be supplied adequately, including the lung, the CNS, the kidney and many more. The undersupplied organs can be damaged, be affected in terms of performance, or even fail. This could have a stronger impact, in particular, on patients with underlying medical conditions, and on elderly people, because lower LysoPC plasma levels tend to occur more frequently in such persons.

Thus, it has been the object of the invention to develop a medicament that is capable of overcoming or at least relieving the LysoPC deficiency caused by the respective disease, and is thus suitable for the treatment, prevention, or support of treatment, and for the aftercare of pneumonias and sepsis, including pneumonia and sepsis as a result of influenza, Covid-19, ARDS, cancer, and pneumonitis, hepatitis, or colitis, after an immune therapy in cancer. In the case of the inflammatory diseases, strengthening of the immune system is to be achieved while damage to organs is avoided, which could currently, in the case of COVID-19, enable the creation of herd immunity while deaths could be avoided.

### SUMMARY OF THE INVENTION

It has been found that there are substantially larger amounts of LysoPC in the body than can be concluded from the LysoPC plasma concentrations. Almost all of the LysoPC in the body, i.e., about 95%, is present in deeper compartments, which are in a fast equilibrium with the plasma compartment. Therefore, the LysoPC conversion rate is higher by more than one order of magnitude than has been assumed before, and at a rough estimate, it is at least 50 g per day.

It has further been found that the LysoPC level can be effectively increased by oral administration of 1,2-acylglycerophosphocholines (PCs). In contrast to the textbook opinion, a virtually complete transition of the orally administered PC as LysoPC into the plasma and the deeper compartments is possible, at least when larger amounts of PC are delivered, the LysoPC formed from PC in the intestine is not degraded in the enterocytes and directly enters the blood.

It has further been found that the LysoPC level can be increased by oral or systemic delivery of alpha-glycerophosphocholine. It seems possible that alpha-GPC is directly converted to LysoPC in the liver by an acylation reaction that has not yet been described.

It has further been found that the LysoPC level can be increased by oral or systemic delivery of glucose. Probably, the glucose is ultimately used in the liver for fatty acid biosynthesis, and the fatty acids formed are then released into the system as LysoPC.

It has further been found that LysoPC could be delivered directly into the blood as an infusion despite its potential hemolytic effects. Since the major part of the LysoPC is not present in the plasma, i.e., not bound to albumin, but is rather present in deeper compartments (tissue) and, in addition, the LysoPC-plasma equilibrium is established very fast, the direct administration of LysoPC appears to be possible. If LysoPC is slowly infused into the blood in large amounts that are therapeutically relevant, it is spontaneously bound to albumin there and thus loses its hemolytic properties. From there, it gets very quickly also into deeper compartments because of the concentration gradient, and the albumin is again available for binding LysoPC and thus for preventing its hemolytic activity.

It has further been found that even muscular tissues show a high consumption of LysoPC, which explains why physical weakness and even heart problems may arise in case of a LysoPC deficiency, e.g., during an influenza.

Finally, it has been found that the build-up of muscle mass by strength training, as is also employed in the therapy of fatigue, was associated with an increase of the LysoPC level. This means that the sports therapy employed in a fatigue can increase a reduced LysoPC level, which is a possible explanation of its success. One possible mechanism is that the trained muscle releases higher amounts of alpha-GPC, so that LysoPC can be increasingly formed in the liver as described above, and this LysoPC in turn transports fatty acids to the muscle more efficiently, where alpha-GPC is then again released. This interpretation fits the discovery described above, namely that the LysoPC level is heavily lowered upon very high physical loads, and the consumption of LysoPC is higher than the provision of LysoPC via alpha-GPC in the liver.

As a consequence of this observation, it is probable that low LysoPC levels and/or low alpha-GPC levels after a cancer disease or inflammatory reaction hinders the restoration of physical fitness, which is manifested by fatigue symptoms. Therefore, it is to be expected that a specific increase of the LysoPC levels, e.g., by the delivery of alpha-GPC to fatigue patients, will lead to a reduction of fatigue symptoms. One facet could be improved muscle build-up under physical workout.

From these results, it has been concluded that the application of phospholipids or alpha-GPC is suitable for reducing or preventing the LysoPC deficiency occurring in viral and bacterial pneumonias, ARDS, sepsis, including pneumonia and sepsis as a result of influenza, Covid-19, ARDS, cancer, pneumonitis, colitis or hepatitis after an immune therapy of cancer, and a fatigue subsequent to the mentioned diseases or treatments. It has further been found that alpha-GPC is a suitable and well applicable LysoPC derivative available at low cost for the treatment and aftercare of cancers and tumor cachexia. Thus, the invention relates to

(1) lyso-phosphatidylcholine (LysoPC), or a suitable precursor or derivative thereof, or a composition containing LysoPC and/or one or more suitable precursors or derivatives thereof, for use in the treatment and aftercare of inflammatory diseases in humans that involve lowering of the LysoPC level, including, in particular, the viral and bacterial pneumonias and sepsis including the prevention or support of treatment and aftercare (e.g., treatment of fatigue) of viral and bacterial pneumonias and sepsis, which includes the treatment of pneumonia and sepsis after influenza, Covid-19, ARDS, and pneumonitis, colitis or hepatitis after an immune therapy of cancer;

(2) a process for the treatment and aftercare of inflammatory diseases in humans accompanied by a lowered LysoPC level, including, in particular, the viral and bacterial pneumonias and sepsis, comprising administering LysoPC or a suitable precursor or derivative thereof, or a composition containing LysoPC and/or one or more suitable precursors or derivatives thereof, to a patient in need of such a treatment or aftercare;

(3) alpha-glycerophosphocholine (alpha-GPC) or a variant thereof (which is of course not LysoPC or PC nor comprises it), or a composition containing alpha-GPC and/or one or more variants thereof, for use in the treatment and aftercare of cancers and tumor cachexia, and for supporting the convalescence after cancer or cancer treatment; and

(4) a process for the treatment and aftercare of cancers and tumor cachexia, and for supporting the convalescence after cancer or cancer treatment, comprising administering alpha-GPC or a variant thereof, or a composition containing alpha-GPC and/or one or more variants thereof, to a patient in need of such a treatment.

Preferred is the delivery of high doses of the composition orally or systemically (i.v.), in order to increase the LysoPC level quickly, and because a higher conversion rate is to be expected in the case of an inflammation or cancer/cachexia.

The invention will be explained further in the following.

### DETAILED DESCRIPTION OF THE INVENTION

The phospholipid-containing composition for oral application according to the invention may contain soy or egg PC, or lecithin, marine phospholipids, e.g., phospholipids from krill or from salmon roe, or from algae. The phospholipids employed may come from various sources or be synthetically produced, wherein the PLs are supposed to contain only a little or no ω-6 fatty acids, but preferably ω-3 fatty acids, such as, e.g., marine phospholipids or krill oil, or ω-9 fatty acids, or saturated fatty acids. They thus contain acylglycerophospholipids (AGPL), especially acylglycerophosphatidylcholines and metabolites thereof, such as lyso-phosphatidylcholine (LysoPC) and alpha-glycerophosphocholine (alpha-GPC), as further defined below:

An “acylglycerophospholipid” (“AGPL”) as used in the present invention includes, e.g., a 1,2-diacylglycerophospholipid, 1-acylglycerophospholipid or 2-acylglycerophospholipid with saturated or unsaturated acyl radicals, including phosphatidylcholine, lyso-phosphatidylcholine, and lecithin, or pharmaceutically suitable salts thereof. AGPL preferably has the structure of formula (I)

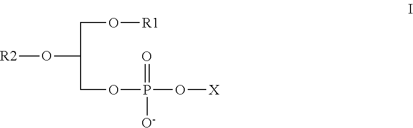

wherein

R¹ and R² are independently selected from H, alkylcarbonyl, alkenylcarbonyl, alkynylcarbonyl, arylalkylcarbonyl and cycloalkylcarbonyl radicals, in which said alkyl radicals can be linear, branched-chain or cyclic, saturated or unsaturated, and substituted with 1 to 3 radicals R³, and in the alkyl radicals, one or more of the carbon atoms may be replaced by O or NR⁴;

X is selected from H (when the compound is a PA), —(CH₂)ₙ-N(R⁴)₃⁺ (a class of compounds comprising PE and PC), —(CH₂)ₙ-CH(N(R⁴)₃⁺)-COO⁻ (a class of compounds comprising PS) and —(CH₂)ₙ-CH(OH)-CH₂OH (a class of compounds comprising PG), wherein n is an integer from 1 to 5;

R³, independently of the occurrence of other R³ radicals, is selected from H, lower alkyl (in which the lower alkyl radicals may be linear, branched-chain or cyclic, saturated or unsaturated), F, Cl, CN and OH; and

R⁴, independently of the occurrence of other R⁴ radicals, is selected from H, CH₃ and CH₂CH₃,

or a pharmacologically suitable salt thereof.

The acyl radicals are preferably alkylcarbonyl radicals. These may be saturated or unsaturated and have identical or different lengths, chain lengths of C10 to C24 being preferred, chain lengths of C14 to C22 being more preferred. Unsaturated alkylcarbonyl radicals are preferably selected from ω-3 and ω-9 fatty acids, especially from oleic acid (18:1), α-linolenic acid (18:3), eicosapentaenoic acid (20:5), and docosahexaenoic acid (22:6).

Preferred for the use according to the invention are AGPLs with naturally occurring head groups, especially phosphatidylcholines, phosphatidylethanolamines, phosphatidylglycerols, phosphatidylserines, and phosphatidic acids, more specifically APGLs selected from the group of phosphatidylcholines.

In a particularly preferred embodiment of (1), compounds having the structure of formula (I) or pharmaceutically suitable salts thereof are used, in which

(i) the alkylcarbonyl radicals have 10 to 24 carbon atoms, are saturated, or contain one or more double bonds, wherein the number of carbon atoms is preferably a multiple of 2, and the double bonds are not conjugated, and wherein the alkyl radicals are more preferably fatty acid radicals;

(ii) the lower alkyl radicals have 1-3 carbon atoms and are preferably saturated; and

(iii) n is an integer from 1 to 3.

Even more preferred are compounds of formula (I) in which

(i) R¹ and R² are independently H or unbranched and unsubstituted alkylcarbonyl radicals, which are either saturated and in this case preferably selected from lauryl (n-dodecanyl), myristyl (n-tetradecanyl), palmitoyl (n-hexadecanyl), stearyl (n-octadecanyl), arachidyl (n-eicosanyl), behenyl (n-docosanyl) and lignoceryl (n-tetracosanyl) radicals, and even more preferably from myristyl, palmitoyl, stearyl and arachidyl radicals, or are unsaturated and in this case preferably selected from oleyl (18:1), α-linolenyl (18:3), eicosapentaenyl (20:5) and docosahexaenyl (22:6) radicals;

(ii) R³ is H; and

(iii) X is —(CH₂-)₂-N(CH₃)₃⁺, —(CH₂-)₂-NH₃⁺, or —CH₂-CH(NH₃⁺)-COO⁻.

The origin of the AGPL (synthetic or isolated from natural sources) is irrelevant to their use according to the invention. Hydrogenated AGPL may also be used. AGPL according to the invention also include lyso-acylglycerophospholipids, which differ from AGPL having two acyl radicals by the fact that one acyl radical is lacking in the sn-1 or sn-2 position. Commercially available mixtures of glycerophospholipids or fractions of such mixtures may also be employed. This is exemplified by so-called lecithin, which must contain at least 20% of phosphatidylcholine. More preferred are AGPL of formula (II)

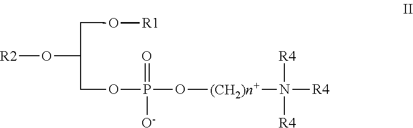

wherein

(i) R¹ and R² are independently H or unbranched and unsubstituted alkylcarbonyl radicals, which are either saturated and in this case preferably selected from lauryl, myristyl, palmitoyl, stearyl, arachidyl, behenyl and lignoceryl radicals, and even more preferably from myristyl, palmitoyl, stearyl and arachidyl radicals, or are unsaturated and in this case preferably selected from oleyl, α-linolenyl, eicosapentaenyl and docosahexaenyl radicals;

(ii) R⁴ is CH₃ or H; and

(iii) n is 2 or 3.

Even more preferred is phosphatidylcholine (PC) with saturated fatty acid radicals, especially dipalmitoylphosphatidylcholine (DPPC). DPPC is contained, inter alia, in lecithin, mainly in hydrogenated lecithin. Among other reasons, this is why lecithin and hydrogenated lecithin are suitable for the use according to the invention. Particularly suitable lecithins include egg or soy lecithin, or hydrogenated egg or soy lecithin.

If the AGPL is a lyso-PL, then the fatty acid is preferably

(i) a fatty acid having a length of at least C16,

(ii) an unsaturated fatty acid, or

(iii) an ω-3 or ω-9 fatty acid, which preferably has at least a length of C18, more preferably C20, and is, in particular, eicosapentaenoic acid (20:5) or docosahexaenoic acid (22:6).

If the AGPL is an alpha-glycerophosphocholine (alpha-GPC), then it is a compound of the above formula (II) in which both radicals R¹ and R² are hydrogen, n is 2, and R⁴ is CH₃. Alpha-GPC is derived from AGPC, but wherein positions 1 and 2 are not substituted. Consequently, alpha-GPC is water-soluble and no longer a lipid.

“Active ingredients” as used in the present invention refers to compounds that are able to provoke a physiological reaction in living organisms, especially in humans or animals. In particular, they are drugs employed in therapy. A “pharmacologically active substance” refers to a compound that, as an ingredient of a medicament, is the cause of its activity.

A “triglyceride” refers to a triester of glycerol with identical or different, saturated or unsaturated acyl radicals having 10 to 30 carbon atoms. Preferred triglycerides for the use according to the invention in combination with the AGPL include fatty acids and those unsaturated fatty acids that may not be converted in vivo to eicosanoids, especially not to the highly biologically active eicosanoids of the 2 series, such as PGE₂. Examples thereof include saturated/hydrogenated triglycerides, MCT, fish oils, or oils from the microalga Ulkenia (Ärztezeitung of Aug. 26, 2004), both of which contain very much EPA and DHA, olive oils, rapeseed oils, evening primrose oils, or linseed oils.

In a preferred embodiment of (1), a 1,2-diacyl-AGPL is the only active ingredient present in the composition, more preferably the only GPL present. In the latter case, it is more preferred that only 1,2-acylglycerophosphocholine is present.

In another preferred embodiment, LysoPC is the only active ingredient contained in the medicament.

In another preferred embodiment, alpha-GPC is the only active ingredient contained in the medicament.

AGPL, LysoPC and alpha-GPC may each be present as the only active ingredient in the medicament, or else as combinations, wherein 1,2-AGPL and alpha-GPC are preferred for oral administration, and LysoPC and alpha-GPC are preferred for systemic (e.g., i.v.) administration.

The individual active ingredients or the combinations may be present as the only active ingredients in the medicament, but they may also be combined with other active ingredients, wherein such other active ingredients may be present in the same composition, or as a separate dosage form.

Combinations of the phospholipid formulations or of alpha-GPC with triglycerides, fatty acid monoesters, waxes or free fatty acids are possible, wherein such lipids should preferably contain anti-inflammatory or at least non-inflammatory fatty acids, i.e., those triglycerides, fatty acid monoesters, waxes or free fatty acids are preferred that contain only a little or no ω-6 fatty acids, but instead ω-3 or ω-9 fatty acids, or saturated fatty acids.

Alpha-GPC (orally or i.v.) can be employed in combination with the PL (see above) or with fish oils, or other oils with suitable fatty acids.

All of the above mentioned active ingredients in combination with: albumin (for systemic administration); with Glucose; optionally with insulin; anti-inflammatory ingredients; antibiotics; proteins/AS for reducing muscle loss (during a disease), e.g., whey protein; and carnitine.

In the context of the present invention, it is important that the phospholipids incorporated into the system are ultimately metabolized, releasing the fatty acids bonded in the phospholipids. Since ω-6 fatty acids, such as arachidonic acid, can be converted to pro-inflammatory eicosanoids, they are avoided in a preferred embodiment of AGPL. AA and its precursor fatty acid linolic acid can be converted to the eicosanoid PGE₂. PGE₂ not only has a pro-inflammatory effect, but it also has an autocrine effect on tumor cells, stimulating their growth, and in addition, an increased PGE₂ level is associated with an increased rate of metastasis (Attiga, F. A. et al., Cancer Res. 60(16): 4629-4637 (2000)). In addition, another important implication of an increased PGE₂ level is pain sensitization, since PGE₂ enhances the excitability of nociceptors (Brune, K. and Zeilhofer, H. U., Biospektrum 1: 36-38 (2004)).

As described above, a lowered LysoPC level in the body caused by a severe inflammatory response or by cancer may possibly damage organs and/or adversely affect their function, or lead to organ failure. Therefore, the effect according to the invention of the mentioned active ingredients is based on the effective substitution of, and support of the neosynthesis of, LysoPC. Since we determined that very much more LysoPC than had been believed before is present in the body, and the body compartments are in a fast equilibrium mutually, and in addition that LysoPC is reduced by other important consumers including not only the lung and the CNS, but also the muscles and the heart, particularly efficient ways of doing this had to be found. Thus, the effect according to the invention of the mentioned active ingredients is coupled to the delivery of sufficient amounts of the active ingredients, in order to ensure a LysoPC quantity in the body that is at least sufficient for the function of the tissues and organs.

The use according to the invention of acylglycerophosphocholines and their metabolites LysoPC and alpha-GPC as active ingredients in a medicament has proven to be a surprisingly effective method for the therapy and support of therapy and for the aftercare of pneumonias, sepsis, influenza, COVID-19, ARDS, or pneumonitis, colitis, or hepatitis after immune therapy and/or the accompanying symptoms of these diseases, including the prevention and support of convalescence, inter alia, by the treatment of fatigue. Thus, the present invention shows, in particular, that the delivery of AGPL and metabolites thereof can support the therapy and aftercare of COVID-19.

To the therapeutic approach for the treatment of pneumonias of different origins and of sepsis as described herein, the determination of the total amount of LysoPC in the body is essential. The latter could be determined by determining the LysoPC concentration in the plasma, which is in equilibrium with the deeper compartments. To this end, one subject was administered, on the one hand, a high amount of phospholipid from egg (Example 1 (Determination of Plasma LysoPC) and Example 2). The plasma volume was calculated, the increase of the LysoPC concentration was determined, and the plasma LysoPC level accompanying this concentration change was calculated. This LysoPC amount and its ratio to the amount taken up yields the distribution of the LysoPC equivalent taken up between the plasma and the deeper compartments, from which the total LysoPC amount in the body can be calculated. It was considered that 100% of the phospholipid is taken up as LysoPC.

In another experiment, a subject was administered a high amount (25 g) of pure triglyceride (Example 3). The plasma volume was calculated. The lowering of the LysoPC concentration because of the consumption of LysoPC for producing PC for the biosynthesis of chylomicrons was determined (One chylomicron contains about 5-10% PC as a ratio to the amount of triglyceride). The amount of plasma LysoPC involved in this concentration change was calculated. This amount of LysoPC and its ratio to the amount taken up yields the distribution of the LysoPC equivalent taken up between the plasma and the deeper compartments, from which the total LysoPC amount in the body can be calculated. It was considered that 100% of the plasma LysoPC was converted to PC.

From the results of Examples 2 and 3, the amount of LysoPC in deeper compartments could be estimated at about 95% of the total body LysoPCs, i.e., about 5% is present in the blood plasma.

Since the subject possessed about 3.5 liters of plasma, and a concentration of 300 μM is assumed (mean value for healthy subjects, see above), a total amount of about 500 mg of LysoPC is present therein. On this basis, the amount of total body LysoPCs can be estimated to a minimum of 10 g.

Based on an estimated total body half life for LysoPC of 2.5 hours (see above), a daily conversion rate of at least 50 g is obtained. Because of this value alone, LysoPC is one of the important energy sources in the body, the glucose conversion rate being about 200 g per day in comparison.

Now, the needs for LysoPC in a healthy person from the currently known LysoPC consumers, i.e., the lung (surfactant production) and the CNS, was estimated not to be sufficient for so high a daily LysoPC conversion rate of at least 50 g. Therefore, the LysoPC needs of the tissue group with the largest proportion, the muscle mass, were examined.

To this end (Example 4), the plasma LysoPC levels directly before and after the run were examined in a group of marathon runners. In all seven runners examined (m/f), the levels declined very clearly, by a maximum of 45%, on average by 28%, which indicates a high LysoPC consumption by the working muscle mass. LysoPC may function as a fatty acid transporter here, which is cleaved again into free fatty acids in the periphery of the muscle cells, whereby a high fatty acid gradient is built up, which is the precondition of a quick uptake of the fatty acids via carnitine. When the fatty acid is released in the muscular tissue, alpha-GPC is released, which, being a water-soluble molecule, can enter the systemic circulation again. The transport of fatty acids using LysoPC as a transport form to cells has been described before in another case, i.e., in the supply of tumor cells with fatty acids. In tumor cells, this is a very strongly expressed supply mechanism, contributing, inter alia, to the low LysoPC levels in cancer patients (see also: Background of the Invention).

In addition, it is known that the heart takes up about 70% of its energy in the form of fatty acids. Whether these fatty acids are also transported to the heart in the form of LysoPC, is unknown, but probable. Therefore, it is to be assumed that damage to the heart is possible in patients with LysoPC levels that are strongly lowered because of an inflammation, because ultimately too little energy is supplied. This is all the more probable as it is observed that many of the people who are currently severely ill from COVID-19 also suffer damage to the heart, and that the pre-existing condition diabetes as well as obesity increase the corresponding risk (Mohammad Madjid et al., JAMA Cardiology, doi: 10.1001/jamacardio.2020.1286; Riccardo Inciardi et al., doi: 10.1001/jamacardio.2020.1096; Shaobo Shi et al., doi:10.1001/jamacardio.2020.0950, Bonow et al., JAMA Cardiology, doi: 10.1001/jamacardio.2020.1105). In diabetes, the uptake of glucose into the heart muscle may be adversely affected. In such a case, if the energy supply by fatty acids is also adversely affected by a LysoPC deficiency into the bargain, this may contribute to a damage to the heart muscle. A possible therapeutic option would be, not only an increase of the LysoPC levels, but also the delivery of glucose and optionally also of insulin for improving glucose uptake.

In Example 4b, it has been shown that the increase of the proportion of muscle mass by a strength training was accompanied by an increase of the LysoPC level. This means that the sports therapy also employed in a fatigue can increase the LysoPC levels that are reduced in a fatigue (Y. Lu et al., J. Clin. Biochem. Nutr. 58: 210-215 (2016)), which is a possible explanation of the success of sports therapy in fatigue. The mechanisms are still unclear, but it is probable that the working muscle not only consumes higher amounts of LysoPC for fatty acid supply, but also releases the “fatty acid carrier” alpha-GPC, whereby LysoPC can be increasingly formed from alpha-GPC in the liver (see Example 5), which in turn transports fatty acids to the muscle, where alpha-GPC is then again released. This is also consistent with the observation described above, that the LysoPC level is strongly lowered at a very high physical load (marathon run) (Example 4a), since the consumption of LysoPC is higher than the provision of LysoPC via alpha-GPC in the liver. In the case of fatigue, such a heavy load like a marathon would lead to a deterioration of the symptoms, which has also been described.

As a consequence of this observation, it is probable that low LysoPC levels and/or low alpha-GPC levels after a cancer disease or inflammatory reaction hinders the restoration of physical fitness, which may be manifested by fatigue symptoms, among other things. Therefore, it is to be expected that a specific increase of the LysoPC levels, e.g., by the delivery of alpha-GPC to fatigue patients, will lead to a reduction of fatigue symptoms.

Although a high daily LysoPC consumption is very plausible because of the knowledge gained herein, according to the explanations in the section “Background of the Invention” and according to the current state of the art, the body is not capable of providing such high daily LysoPC amounts of at least 50 g.

Therefore, it has been tested whether the alpha-GPC released from LysoPC in the cleaving of fatty acids can be reacylated again, possibly in the liver as the site of fatty acid biosynthesis and as a known source of LysoPC, at least if albumin is present for binding the LysoPC from the hepatocytes outside the cell (Baisted, DJ. et al. Biochem. J. 253: 693-701 (1988)). Such a reacylation reaction of alpha-GPC has not been described for humans and animals, but it has for yeasts (Saccharomyces cerevisiae) (Stalberg, K. et al., J. Lipid Res. 49: 1794-1806 (2008); Anaokar, S. et al., J. Biol. Chem. 25; 294(4): 1189-1201 (2018)).

Example 5 shows that the oral uptake of 600 mg of alpha-GPC leads to an increase of the amount of LysoPC in the body in a subject, which suggests the above mentioned reaction and indicates a way of how the necessary high amounts of LysoPC can be provided in the body.

In another experiment (Example 6), it was tried to enhance the amount of LysoPC by increasing the fatty acid biosynthesis in the liver. For this purpose, a subject ingested 50 g of glucose, which also resulted in a quick increase of the LysoPC plasma level.

Thus, the effect according to the invention of the AGPL and alpha-GPC in the treatment, prevention, support of treatment, and aftercare (convalescence) of pneumonias, sepsis, influenza, COVID-19, ARDS, cancer including a fatigue following the basic disease, or pneumonitis, colitis, or hepatitis after immune therapy in cancer is probably explained by the fact that the substances at least in part compensate for the strong loss of LysoPC associated with such diseases, and thus can enhance the immune defense, on the other hand prevent the immune response from exacerbating, and ensure the supply of important organs and muscular tissue with LysoPC, especially the lung and the heart.

Further, the effect according to the invention of the AGPL and the alpha-GPC is presumably based on the fact that large fractions of the LysoPCs supplied or formed in the body are ultimately cleaved again in vivo into fatty acids and alpha-GPC. Then, these two components additionally take an effect individually or together that could not be achieved without problems by administering one of the two components alone. Thus, alpha-GPC can be reacylated to LysoPC again, and thus provides for a longer-term stabilization of the LysoPC household, beyond the initial substitution.

Because of what has been said above, one even more preferred embodiment is the use of AGPL (PC or LysoPC), which preferably predominantly or exclusively contain hydrogenated ω-3 or ω-9 fatty acids as fatty acids, and do not, or only in low fractions, contain ω-6 fatty acids. In other words: they predominantly or exclusively contain fatty acid radicals that cannot be converted in vivo to the highly biologically active pro-inflammatory eicosanoids, e.g., prostaglandin E2. These AGPL may be, inter alia, ω-3-containing phospholipids, such as those obtained from salmon roe or krill, for example. Also important are hydrogenated phospholipids or LysoPCs, which are obtained by the hydrogenation of phospholipids from natural resources, e.g., from egg or soybean. Synthetically produced phosphatidylcholines, such as DPPC, are also possible.

One even more preferred embodiment is the use according to the invention of the AGPL and alpha-GPC in combination with a triglyceride or the fatty acids, mono- and diglycerides that can be prepared from a triglyceride, or of free fatty acids, waxes, or fatty acid monoesters. Of these, the use of the triglyceride as a further ingredient of the medicament is preferred. Said compounds preferably predominantly or exclusively are or contain hydrogenated ω-3 or ω-9 fatty acids, and they do not, or only in low fractions, contain ω-6 fatty acids. In other words: they predominantly or exclusively contain or are fatty acid radicals that cannot be converted in vivo to the highly biologically active pro-inflammatory eicosanoids, e.g., prostaglandin E2. Such triglycerides or the resulting di- and monoglycerides and fatty acids are, for example, saturated/hydrogenated triglycerides, MCT, fish oils, or oils from the microalga Ulkenia (Ärztezeitung of Aug. 26, 2004), both of which contain very much EPA and DHA, olive oils, rapeseed oils, evening primrose oils, or linseed oils.

The activity of the medicament, especially the effect on the inflammation to be treated, can be enhanced thereby. On the one hand, the cause of this effect is the known activity of triglycerides as high energy food and energy supplier. On the other hand, an additional effect results from the simultaneous presence of the triglycerides and the AGPL or the alpha-GPC because, for example, the saturated ω-3 or ω-9 fatty acids additionally applied as triglycerides become components of the ultimately formed LysoPCs. This can happen because the fatty acids of the triglycerides are released in the GI tract, and also the AGPL are at least in part reconstructed in the enterocytes, which is why fatty acids from oils can then be incorporated into the newly synthesized AGPL. Thus, it is conceivable that, instead of the ω-3-containing marine phospholipids whose resources are limited and which are expensive, a mixture of AGPL from, e.g., soybean or egg is delivered in combination with a ω-3-containing fish oil whose resources are not so much limited. Further, the delivery of corresponding oils may require an increased presence of saturated as well as ω-3 and ω-9 fatty acids in the liver, whereby a LysoPC with just these fatty acids can then be formed easily from alpha-GPC.

Further, said saturated or ω-3 or ω-9 fatty acids are of course also distributed in the body through the ordinary lipid metabolism and thus can exhibit their potentially anti-inflammatory activity in different organs or tissues, and thus act synergistically with the medicaments defined herein.

The essential point in the use according to the invention of the AGPL and alpha-GPC in combination with one or more triglycerides is the fact that the AGPL proportion of the total lipid content of the medicament is high. Thus, this AGPL proportion should be at least 10% by weight, but preferably at least 20% by weight, even more preferably at least 40% by weight of the total lipid weight. This is equivalent to a ratio of AGPL:triglyceride of 1:9, preferably 2:8, more preferably 4:6. This is because the AGPL as active ingredients are merely supported in their activity by the presence of the triglycerides. This is also the difference between the medicaments of the present invention and lipid preparations for artificial feeding, in which the PL proportion must be kept as low as possible, and oils with high contents of ω-3 or ω-6 fatty acids, whose intake especially aims at an increase of the mentioned fatty acid levels in the body. Further, the AGPL in combinations with triglycerides can contain a wide range of fatty acids (hydrogenated, unsaturated) and originate from a wide variety of sources (egg, soybean, fish etc.), which means that they are not limited to egg lecithin like the formulations for artificial nutrition.

If LysoPC, as a further embodiment, is delivered, not orally, but systemically, especially i.v., then this is preferably done as a permanent infusion in order to avoid high peak concentrations in the blood, which could lead to hemolysis. In order to further reduce this problem, it is preferred to deliver LysoPC also in combination with albumin, at least in the first infusions, in order to ensure that the albumin level in the patients is not lowered, which would again increase the risk of hemolysis. In addition, it is known that the presence of albumin is necessary for the release of LysoPC from the liver.

Further, it is possible to additionally deliver glucose to the patients (i.v. or orally), for the fatty acid biosynthesis in the liver to be stimulated, which, in combination with the delivery of alpha-GPC or AGPL (which are ultimately converted to alpha-GPC), may lead to an enhanced intrinsic LysoPC synthesis.

For the fatty acids that are possibly released in the periphery of the cells to be able to easily enter the cells, a further embodiment of the invention is the combination of a delivery of AGPL and/or alpha-GPC with carnitine, which could contribute to an improvement of the fatty acid transport in the cells.

In addition to the effects for increasing the LysoPC level by the measures mentioned herein and the accompanying improved energy supply to the heart tissue and other organs, it is possible, especially for diabetics, to also deliver insulin, so that the optimized cellular provision of glucose can additionally enhance the effects of LysoPC.

Another even more preferred embodiment is the use according to the invention of the AGPL in combination with substances having an effect on eicosanoid synthesis, thus being able to support the effect of AGPL synergistically. Firstly, these are PLA2 inhibitors, especially inhibitors of sPLA2 (type II sPLA as described, e.g., in Uhl et al., Phospholipase A2 Basic and Clinical Aspects in Inflammatory Diseases Vol. 24, Karger, Basel, pp. 123-175 (1997), and in DE-A-4234130).

Secondly, these are substances that inhibit the cyclooxygenases 1, 2 or 3, such as the non-specific compounds acetylsalicylic acid, paracetamol, diclofenac, ibuprofen, metamizole, phenazone, propyphenazone, and COX-2 inhibitors, e.g., meloxicam (Mobec®), celecoxib (Celebrex®), rofecoxib (Vioxx®), etoricoxib (Arcoxia®), valdecoxib (Rayzon®), and parecoxib (Dynastat®).

Further, these are substances that can prevent the virus from spreading in the case of viral infections, i.e., virostatics, or substances that can prevent the bacteria from spreading in the case of bacterial infections, i.e., antibiotics.

These substances are employed in concentrations/doses as usual for the therapeutic use of the substances, or prescribed by the manufacturer.

The compositions according to the invention are suitable for systemic administration, especially for oral (p.o.) or intravenous (i.v.) administration.

The composition according to the invention may be in the form of an emulsion or solution, tablets, capsules, or powder, e.g., to be stirred into foods. In the preferred formulation as a tablet, capsule or powder, the concentration of the AGPL may be up to 100%. In the case of alpha-GPC or a variant thereof, solutions or aqueous solutions are preferred for the administration because of the high solubility of such compounds.

In addition to the phospholipids, alpha-GPC and other active ingredients, the composition according to the invention may also contain usual pharmaceutically acceptable vehicles, excipients, stabilizers, diluents, binders etc.

In addition to the alpha-GPC or derivatives thereof, the composition according to the invention for the treatment and aftercare of cancers and tumor cachexia may also contain the above defined LysoPCs or suitable precursors or derivatives thereof, and the mentioned further active ingredients and usual pharmaceutically acceptable vehicles, excipients, stabilizers, diluents, binders etc.

The dosage of the composition is adapted to the respective patient individually by the attending physician. It depends, inter alia, on the kind of disease, the severeness of the symptoms to be treated, the constitution of the patient, etc., wherein doses of 2-300 mg AGPL/kg of body weight per day and at least 500 mg of alpha-GPC are usually considered, a dose of at least 20 mg of AGPL/kg of body weight per day being preferred. In a particularly preferred embodiment, a determination of the LysoPC level in the respective patient is performed to determine the optimum dosage.

Further preferred is a high-dose treatment of the inflammatory diseases with the LysoPC and its derivatives and precursors, wherein the dose is (a) preferably larger than about 2 g of PC/day, more preferably larger than 6 g of PC/day, (b) larger than 1.3 g of LysoPC/day, more preferably larger than 4 g of LysoPC/day, and (c) larger than 0.6 g of alpha-GPC/day, more preferably larger than 2 g of alpha-GPC/day.

A particular advantage of the composition according to the invention resides in its importance to the production of medicaments for the treatment, prevention or support of treatment and aftercare (convalescence) of pneumonias, sepsis, influenza, Covid-19, ARDS, or for supporting immunotherapy in cancer in view of effectiveness and for reducing side-effects, such as pneumonitis, colitis or hepatitis, and for reducing undesirable vaccination responses. In the case of compositions containing alpha-GPC or a variant thereof, this also pertains to the aftercare of cancers, especially cachexia and fatigue.

The invention is further illustrated by means of the following Examples, which do not limit the invention, however.

### EXAMPLES

### Example 1: Assaying of LysoPC in the Plasma, Serum or Blood

LysoPC from plasma, serum or blood was extracted in one step by a newly developed method and subsequently assayed by using HPTLC (high performance thin layer chromatography).

The extraction of 50 μl of sample each was effected using salt-assisted extraction. For this purpose, a ceramic sphere (3.4 mm, ceramic) was charged in a 2 ml twist-top vial, and then 50 μl of distilled water, 50 μl of sample, 192 μl of concentrated ammonium acetate solution, and 300 μl of acetonitrile/1-pentanol (2.5:1 v/v) were added. After closing, the vial is intensively shaken centrifugally using dual centrifugation (ZentriMix 380 R, from the company Hettich, Tuttlingen, Germany) at 2000 rpm and 20° C. for 4 min. Subsequently, the vials are centrifuged at 16,000 rpm for 10 min. From the upper phase, 200 μl is removed, transferred to a crimp-top vial with a microinsert, crimped, and sent to HPTLC.

For the determination of the LysoPC concentrations using HPTLC, five LysoPC standards (16, 40, 80, 120 and 160 μM LysoPC in acetonitrile/1-pentanol (2.5:1 v/v.) and up to 12 sample extracts were applied to a 20×10 cm silica gel plate using the automated applying device ATS4 (CAMAG, Switzerland). The volume applied is 6 μl each.

After the application and after a dwell time of five minutes to ensure uniform evaporation of the solvent, the plate is placed into a preconditioned DC chamber filled with 35 ml of chloroform/methanol/water (65:35:4 v/v) and 200 μl of 25% ammonia solution for 20 min. Within this period, a travelling distance of about 9 cm is reached. Subsequently, in order to evaporate the mobile solvent, the plate is dried on a thermal board at 150° C. and dipped into a copper sulfate solution (83.3 g of copper sulfate pentahydrate and 66.6 ml of phosphoric acid (85%), ad 1000 ml of water) three times for two seconds each. Subsequently, the back side of the plate is blotted dry, and the plate is developed in a preheated drying cabinet at 160° C. for 10 min. After cooling to room temperature again, the optical density of the LysoPC bands (Rf about 0.1) is determined using the evaluation unit (TLC Visualizer 2, CAMAG, Switzerland), and the LysoPC concentrations were evaluated by comparing with the calibration substances using the HPTLC software “visionCATS” (Camag, Switzerland). If the LysoPC value is determined, not directly from plasma, but from whole blood, then the value needs to be corrected by the respective hematocrit value and by a factor of 1.3. The interassay precision of the determination can be described as “good”, with a COV of 11.3%.

### Example 2: Assaying the Total Amount of LysoPC in the Body

A healthy subject (male, 57 years old, 84 kg) with a plasma volume of about 3.5 liters in a fasted state was administered a total of 5 cooked eggs of 60 g each, and subsequently, 50 μl each of blood is sampled from the fingertip over 3 hours at intervals of 15 or 30 min. The determination of the blood LysoPC concentration was effected as described in Example 1.

The determination of the plasma LysoPC concentration in a fasted state yielded 190 μM. After ingestion of 5 eggs with a total of 86.5 g of egg yolk, which contains about 5.8 g of phosphatidylcholine and 360 mg of LysoPC (Blesso, CH., Nutrients 7: 2731-2747 (2015)), the LysoPC level in the plasma rises quickly and reached its peak after 75 min with an increase of the LysoPC level by 70%, and thereafter, it remained on a constant level of about 60-70% above the starting LysoPC level until the end of the measurement.

Since about 330 mg of LysoPC was present in the 3.5 liters of plasma with an initial value of 190 μM at the beginning of the measurements, a rise of the level by 70% corresponds to an additional amount of LysoPC of about 230 mg in the plasma.

When it is assumed that the total phosphatidylcholine supplied by the eggs is cleaved in the GI tract to form LysoPC, a maximum LysoPC uptake of 4,300 mg is obtained together with the LysoPC that is contained in the egg anyway.

Thus, of the supplied amount of LysoPC of 4,300 mg, only 230 mg was found again in the plasma (5.3%). In a first coarse approximation, it can be concluded therefrom that the majority (about 95%) of the LysoPC supplied is located in deeper compartments, and that the plasma compartment and the deeper compartments (organs, tissues) are in a fast equilibrium.

### Example 3: Assaying the Total Amount of LysoPC in the Body

A healthy subject (male, 57 years old, 84 kg) with a plasma volume of about 3.5 liters in a fasted state was administered 25 g of pure triglycerides (margarine), and subsequently, 50 μl each of blood is sampled from the fingertip over 3 hours at intervals of 15 or 30 min. The determination of the blood LysoPC concentration was effected as described in Example 1.

The determination of the plasma LysoPC concentration in a fasted state again yielded 190 μM. After ingestion of the triglycerides, the LysoPC level dropped almost linearly and reached the strongest decrease after about 2 hours (about—20%), and thereafter remained on a constant level of about 20% below the starting LysoPC level until the end of the measurement.

This experiment was performed because the result in Example 2 could also be explained by assuming that only a very small proportion of the LysoPC formed from the PC in the GI tract is ultimately transferred into deeper compartments, and only a low proportion gets into the blood, while the majority was degraded by the enterocytes. In order to show that this is not the case, pure triglyceride was supplied, which ultimately gets into the blood as a chylomicron via the lymphatic system. In addition to the apo-lipoproteins and triglycerides, the construction of chylomicrons requires about 10% phospholipids, which must be synthesized by the enterocytes. For this, the reacylation of LysoPC from the plasma suggests itself as a very fast synthetic pathway.

Since about 330 mg of LysoPC was present in the 3.5 liters of plasma with an initial value of 190 μM at the beginning of the measurements, the dropping of the level by 20% corresponds to a loss of about 66 mg of LysoPC in the plasma.

When it is assumed that all of the triglyceride supplied was converted to chylomicrons in the enterocytes, and that chylomicrons consist of up to 10% of phospholipids, mostly phosphatidylcholine (Stein, Y., Lipid-Review 5: 38 (1988)), then a PC requirement for preparing the chylomicrons of up to 2.5 g is obtained. If this amount of PC is synthesized through the fast reacylation of LysoPC, then this corresponds to a LysoPC removal from the system of 1700 mg.

Of the consumed amount of LysoPC of 1700 mg, about 66 mg was taken from the plasma (3.9%). In a first coarse approximation, it can be concluded therefrom that the majority (about 96%) of the LysoPC consumed by the process of chylomicron synthesis was taken from deeper compartments, and that about 96% of the total body LysoPC is located in such deeper compartments, and that the plasma compartment and the deeper compartments (organs, tissues) are in a fast equilibrium.

From the results of Examples 2 and 3, the amount of LysoPC in deeper compartments could be estimated at about 95-96% of the total body LysoPCs, i.e., 4-5% is located in the blood plasma.

Converted to a mean LysoPC value of 300 μM in healthy subjects (see above) and a plasma volume of about 3.5 liters, about 500 mg of LysoPC is located in the plasma. On this basis, the amount of total body LysoPCs can be estimated coarsely to about 10 g.

Based on an estimated total body half life for LysoPC of 2.5 hours (see above), a daily conversion rate of about 50 g in healthy subjects is obtained, while this value should be significantly higher in pathological situations, such as cancer or an inflammation (see above).

### Example 4: Determination of the Change of LysoPC Concentrations as a Consequence of Physical Activity

Example 4a: Assaying the LysoPC Concentration in Endurance Performance

In a group of 8 subjects (male), the plasma LysoPC levels were examined directly before and after a marathon run. The values before the run were at 294,9 μM on average (222.3-367.9 μM). Because of the run, the levels dropped to 213.6 μM on average, i.e., by 27.6% (156.3-258.4 μM), the strongest decrease being −150 μM (−45%). The LysoPC drop measured here can be explained by a very high consumption from the enduring physical performance, and by the fact that the extra consumption of LysoPC necessary for the athletic performance can no longer be compensated by the normal mechanisms of LysoPC biosynthesis.

Example 4b: Assaying the LysoPC Concentration Before and After 3 Months of Strength Training

In a group of 20 subjects (male, healthy, 35-64 years old), the plasma LysoPC levels were examined before and after 3 months of strength training (3 times a week). The values before the training were at 333.6+−95.9 μM on average, and at 375+−97.5 μM after the training, resulting in an increase of 15.7% (−27.5−+57.2%, p=0.015) on average. The mean fat-free mass increased by 11.7% (10.5-34.6%), by 6.6 kg on average. The LysoPC values after the end of the training correlate significantly with the relative increase of muscle mass (gain of muscle mass/body weight) with an R² of 0.77.

### Example 5: Determination of the Increase of LysoPC Level from the Oral Ingestion of Alpha-Glycerophosphocholine

Since, when a fatty acid is released from LysoPC in the tissue, the water-soluble alpha-glycerophosphocholine is released at the same time, the latter is left behind in aqueous body compartments. In order to test whether alpha-GPC could also be available as an acceptor for the uptake of fatty acids, e.g., in the liver, the change of the LysoPC level after the uptake of 600 mg of alpha-GPC was determined.

A healthy subject (male, 57 years old, 84 kg) with a plasma volume of about 3.5 liters in a fasted state was administered 600 mg of alpha-GPC, and subsequently, 50 μl each of blood is sampled from the fingertip over 2 hours at intervals of 15 or 30 min. The determination of the blood LysoPC concentration was effected as described in Example 1.

The determination of the plasma LysoPC concentration in a fasted state again yielded about 190 μM. The LysoPC level did not change in the first hour after the uptake of alpha-GPC, but then increased in the next 30 min by about 20%, and remained constantly at this value until the end of the measurement.

Since about 330 mg of LysoPC was present in the 3.5 liters of plasma with an initial value of 190 μM at the beginning of the measurements, a rise of the level by 20% corresponds to an additional amount of LysoPC of about 66 mg in the plasma.

When it is assumed that only about 5% of the total body LysoPCs are in the plasma, the increase in the plasma of about 66 mg means a total increase of about 1,300 mg of LysoPC, or 2.6 mmol of LysoPC (MR=about 500 g/mol). This corresponds very well to to molar quantity, namely 2.3 mmol, of the orally ingested alpha-GPC (MR=257.2 g/mol), which provides a first indication that large proportions of additionally ingested alpha-GPC is actually converted again to LysoPC. This shows that alpha-GPC or LysoPC could be an important systemic fatty acid carrier.

### Example 6: Determination of the Increase of LysoPC Level from the Oral Ingestion of Glucose

Since the ingestion of glucose also leads to an increase of the fatty acid biosynthesis in the liver, inter alia, it was tested in this experiment whether this additional fatty acid biosynthesis is associated with an increase of the amount of LysoPC in the body.

A healthy subject (male, 57 years old, 84 kg) with a plasma volume of about 3.5 liters in a fasted state was administered 50 g of glucose, and subsequently, 50 μl each of blood is sampled from the fingertip at intervals of 15 or 30 min. The determination of the blood LysoPC concentration was effected as described in Example 1.

The determination of the plasma LysoPC concentration in a fasted state again yielded about 190 μM. The LysoPC level increased by about 35% over 2 hours, and later measuring points were not available.

Since about 330 mg of LysoPC was present in the 3.5 liters of plasma with an initial value of 190 μM at the beginning of the measurements, a rise of the level by 35% corresponds to an additional amount of LysoPC of about 116 mg in the plasma.

When it is assumed that only about 5% of the total body LysoPCs are in the plasma, the increase in the plasma of about 116 mg means a total increase of about 2,300 mg of LysoPC, or 4.6 mmol of LysoPC (MR=about 500 g/mol).

This shows that it can also be possible that the provision of fatty acids in the liver induces an increase of the LysoPC biosynthesis. In particular, this result again indicates that LysoPC could be an important fatty acid carrier.

### Example 7: Comparison of LysoPC Levels Over Several Days

The LysoPC levels determined in Examples 2, 3, 4 and 6 were acquired over a period of 1 week with always the same healthy subject (male, 57 years, 84 kg) with a plasma volume of about 3.5 liters. The blood samples for the initial values were all taken in the morning at about 9 a.m. in a fasted state (food abstinence began in the previous evening at about 7 p.m.).

Surprisingly, the initial values obtained were always at 190 μM LysoPC, variations were not observed. This shows that the individual LysoPC levels can be individually different at least for similar life styles, but in turn vary only very little for a particular human.

1. Lyso-phosphatidylcholine (LysoPC), or a suitable precursor or derivative thereof, or a composition containing LysoPC and/or one or more suitable precursors or derivatives thereof, for use in the treatment and aftercare of inflammatory diseases in humans that involve lowering of the LysoPC level.

2. LysoPC or a suitable precursor or derivative thereof, or a composition containing LysoPC and/or one or more suitable precursors or derivatives thereof, for use in the treatment and aftercare of inflammatory diseases according to claim 1, which
(i) is used for the treatment of viral and bacterial pneumonias and sepsis; or
(ii) is used for the treatment, prevention, or support of treatment, and for aftercare of viral and bacterial pneumonias and sepsis, including influenza, Covid-19, ARDS, cancer, and support of immune therapy in cancer; or
(iii) is used for the treatment, prevention, support of aftercare of acute and chronic inflammatory diseases and infections that involve lowering of the LysoPC level, especially the treatment and aftercare of sepsis, Covid-19, bowel inflammations, such as Crohn's disease, and colitis ulcerosa, ARDS, COPD, pneumonias (viral, idiopathic or bacterial), inflammatory liver diseases, such as ASH, NASH including alcoholic and non-alcoholic steatohepatitis, hepatitis, acute bacterial and viral infections, vessel inflammations, bladder inflammation (acute and chronic), pyelitis, tendonitis, acute tooth inflammations, sinusitis, abscesses, the support of immune therapy of cancer, for improving the effectiveness and reducing side effects, such as pneumonitis, colitis or hepatitis, for reducing fatigue during and after the above mentioned diseases, and for reducing adverse vaccination responses.

3. LysoPC or a suitable precursor or derivative thereof, or a composition containing LysoPC and/or one or more suitable precursors or derivatives thereof, for use in the treatment and aftercare of inflammatory diseases according to claim 1 or 2, wherein said LysoPC or suitable precursor or derivative thereof is one of the following compounds, or the composition contains one or more of these compounds:
(i) acylglycerophospholipids selected from 1,2-diacylglycerophospholipids, 1-acylglycerophospholipids and 2-acylglycerophospholipids with saturated or unsaturated acyl radicals, including phosphatidylcholines, lyso-phos-phatidylcholines and lecithin, wherein these compounds are synthetically prepared, or originate from natural sources, and are preferably (a) lecithin, more preferably hydrogenated lecithin, and/or (b) a phosphatidylcholine, more preferably dipalmitoylphosphatidylcholine; and/or
(ii) acylglycerophospholipids having a structure of formula (I) 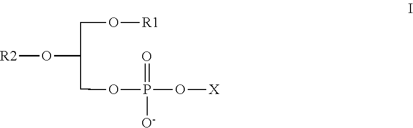
wherein
R¹ and R² are independently selected from H, alkylcarbonyl, alkenylcarbonyl, alkynylcarbonyl, arylalkylcarbonyl and cycloalkylcarbonyl radicals, in which said alkyl radicals can be linear, branched-chain or cyclic, saturated or unsaturated, and substituted with 1 to 3 radicals R³, and in the alkyl radicals, one or more of the carbon atoms may be replaced by O or NR⁴;
X is selected from H, —(CH₂)ₙ-N(R⁴)₃⁺, —(CH₂)ₙ-CH(N(R⁴)₃⁺)-COO⁻ and —(CH₂)ₙ-CH(OH)-CH₂OH, wherein n is an integer from 1 to 5; R³, independently of the occurrence of other R³ radicals, is selected from H, lower alkyl (in which the lower alkyl radicals may be linear, branched-chain or cyclic, saturated or unsaturated), F, Cl, CN and OH; and R⁴, independently of the occurrence of other R⁴ radicals, is selected from H, CH₃ and CH₂CH₃,
or pharmaceutically suitable salts thereof; and/or
(iii) phosphatidylcholines of formula (II) 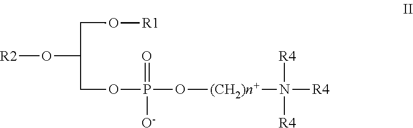
wherein
R¹ and R² are independently H or unbranched and unsubstituted alkylcarbonyl radicals, which are either saturated and in this case preferably selected from lauryl, myristyl, palmitoyl, stearyl, arachidyl, behenyl and lignoceryl radicals, and even more preferably from myristyl, palmitoyl, stearyl and arachidyl radicals, or are unsaturated and in this case preferably selected from oleyl, α-linolenyl, eicosapentaenyl and docosahexaenyl radicals;
R⁴ are independently CH₃ or H, and
n is 2 or 3; and/or
(iv) lyso-phospholipids, compounds of the above compounds of formulas (I) and (II) in which one of R¹ and R² is H; and/or
(v) alpha-glycerophosphocholine (alpha-GPC), a compound of formula (II) in which R¹ and R² are both H.

4. LysoPC or a suitable precursor or derivative thereof, or a composition containing LysoPC and/or one or more suitable precursors or derivatives thereof, for use in the treatment and aftercare of inflammatory diseases according to claim 3, wherein in the compound of formula (I) the alkylcarbonyl radicals have 10 to 24 carbon atoms, are saturated, or contain one or more double bonds, wherein the number of carbon atoms is preferably a multiple of 2, and the double bonds are not conjugated, and wherein the alkyl radicals are more preferably fatty acid radicals; the lower alkyl radicals have 1-3 carbon atoms and are preferably saturated; and n is an integer from 1 to 3.

5. LysoPC or a suitable precursor or derivative thereof, or a composition containing LysoPC and/or one or more suitable precursors or derivatives thereof, for use in the treatment and aftercare of inflammatory diseases according to claim 3, wherein in the compound of formula (I) R¹ and R² are independently H or unbranched and unsubstituted alkylcarbonyl radicals, which are either saturated and in this case preferably selected from lauryl (n-dodecanyl), myristyl (n-tetradecanyl), palmitoyl (n-hexadecanyl), stearyl (n-octadecanyl), arachidyl (n-eicosanyl), behenyl (n-docosanyl) and lignoceryl (n-tetracosanyl) radicals, and even more preferably from myristyl, palmitoyl, stearyl and arachidyl radicals, or are unsaturated and in this case preferably selected from oleyl (18:1), α-linolenyl (18:3), eicosapentaenyl (20:5) and docosahexaenyl (22:6) radicals; R³ is H; and X is —(CH₂)₂-N(CH₃)₃⁺, —(CH₂)₂-NH₃⁺, or —CH₂-CH(NH₃⁺)-COO⁻.

6. LysoPC or a suitable precursor or derivative thereof, or a composition containing LysoPC and/or one or more suitable precursors or derivatives thereof, for use in the treatment and aftercare of inflammatory diseases according to claim 3, wherein said phosphatidylcholine of formula (II) is a phosphatidylcholine (PC) with saturated fatty acid radicals, especially dipalmitoylphosphatidylcholine (DPPC), and more preferably, is egg or soy lecithin, or hydrogenated egg or soy lecithin.

7. LysoPC or a suitable precursor or derivative thereof, or a composition containing LysoPC and/or one or more suitable precursors or derivatives thereof, for use in the treatment and aftercare of inflammatory diseases according to claim 3, wherein said lyso-phospholipid is
(i) a fatty acid having a length of at least C16,
(ii) an unsaturated fatty acid, or
(iii) an ω-3 or ω-9 fatty acid, which preferably has at least a length of C18, more preferably C20, and is, in particular, eicosapentaenoic acid (20:5), or docosahexaenoic acid (22:6).

8. LysoPC or a suitable precursor or derivative thereof, or a composition containing LysoPC and/or one or more suitable precursors or derivatives thereof, for use in the treatment and aftercare of inflammatory diseases according to one or more of claims 1 to 7, wherein the proportion of the phospholipid or acylglycerophospholipid to the total lipids in the composition is at least 10% by weight, preferably at least 40% by weight, even more preferably 100% by weight.

9. LysoPC or a suitable precursor or derivative thereof, or a composition containing LysoPC and/or one or more suitable precursors or derivatives thereof, for use in the treatment and aftercare of inflammatory diseases according to one or more of claims 1 to 8, wherein
(i) said acylglycerophospholipid is the only pharmacologically active ingredient present in the composition, more preferably the only GPL present, wherein, in particular, only acylglycerophosphocholine is present; or
(ii) LysoPC is the only phospholipid contained in the composition; or
(iii) alpha-GPC is the only active ingredient contained in the composition; or
(iv) GPL, LysoPC and alpha-GPC are present in the composition as combinations, preferably (a) AGPL and alpha-GPC (orally), (b) LysoPC and alpha-GPC (systemically).

10. LysoPC or a suitable precursor or derivative thereof, or a composition containing LysoPC and/or one or more suitable precursors or derivatives thereof, for use in the treatment and aftercare of inflammatory diseases according to one or more of claims 1 to 8, wherein said composition further contains
(i) triglycerides, fatty acid monoesters, waxes, and/or free fatty acids, wherein such lipids preferably contain anti-inflammatory and/or non-inflammatory fatty acids, more preferably oils containing little or no ω-6 fatty acids, but instead ω-3 or ω-9 fatty acids, or saturated fatty acids; or
(ii) alpha-GPC (orally or i.v.); or
(iii) alpha-GPC (orally or i.v.) in combination with the PL (see above) or with fish oils, or other oils with suitable fatty acids.

11. LysoPC or a suitable precursor or derivative thereof, or a composition containing LysoPC and/or one or more suitable precursors or derivatives thereof, for use in the treatment and aftercare of inflammatory diseases according to claim 10, wherein the compositions further contain one or more of the following active ingredients: albumin (when administered systemically); glucose; insulin; anti-inflammatory agents, antibiotics; antivirally active substances; and proteins/AA to reduce muscle loss, such as whey protein and carnitine.

12. LysoPC or a suitable precursor or derivative thereof, or a composition containing LysoPC and/or one or more suitable precursors or derivatives thereof, for use in the treatment and aftercare of inflammatory diseases according to one or more of claims 1 to 11, wherein said composition
(i) is suitable for oral or intravenous administration; and/or
(ii) is suitable for the administration of doses of 2-300 mg of AGPL/kg of body weight, or at least 500 mg of alpha-GPC per day, preferably at least 20 mg of AGPL/kg of body weight per day; and/or
(iii) is used for the high dosage treatment of the inflammatory diseases, wherein the dose is (a) preferably larger than about 2 g of PC/day, more preferably larger than 6 g of PC/day, (b) larger than 1.3 g of LysoPC/day, more preferably larger than 4 g of LysoPC/day, and (c) larger than 0.6 g of alpha-GPC/day, more preferably larger than 2 g of alpha-GPC/day.

13. LysoPC or a suitable precursor or derivative thereof, or a composition containing LysoPC and/or one or more suitable precursors or derivatives thereof, for use in the treatment and aftercare of inflammatory diseases according to one or more of claims 1 to 12, wherein the composition is in the form of an emulsion or solution, tablets, capsules, or powder, to be stirred into foods, or for direct ingestion.

14. Use of LysoPC or of a suitable precursor or derivative thereof, or of a composition containing LysoPC and/or one or more suitable precursors or derivatives thereof, for preparing a medicament for the treatment and aftercare of inflammatory diseases in humans that are accompanied by a lowered LysoPC level.

15. A process for the treatment or aftercare of inflammatory diseases in humans accompanied by a lowered LysoPC level, comprising administering LysoPC or a suitable precursor or derivative thereof, or a composition containing LysoPC and/or one or more suitable precursors or derivatives thereof, to a patient in need of such a treatment or aftercare.

16. Alpha-glycerophosphocholine (alpha-GPC), or a variant thereof, or a composition containing alpha-GPC and/or one or more variants thereof, for use in the treatment of cancers and tumor cachexia, and for supporting the convalescence after cancer or cancer treatment in humans.

17. Alpha-GPC, or a variant thereof, or a composition containing alpha-GPC and/or one or more variants thereof, for use in the treatment of cancers and tumor cachexia, and for supporting the convalescence after cancer or cancer treatment according to claim 16, wherein said alpha-GPC or variant thereof is a compound of formula (II) 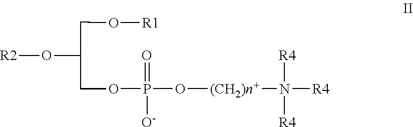
wherein R¹ and R² are both H, R⁴ are independently CH₃ or H, and n is 2 or 3.

18. Alpha-GPC, or a derivative thereof, or a composition containing alpha-GPC and/or one or more derivatives thereof, for use in the treatment of cancers and tumor cachexia, and for supporting the convalescence after cancer or cancer treatment according to claim 16 or 17, wherein said cancers and tumor cachexia include solid tumors, especially metastatic tumors, sarcomas, hematological tumors, GI tumors, endocrine tumors, tumors of the CNS, brain tumors, female tumors, male tumors, breast cancer, prostate carcinoma, colon carcinoma, lung cancer, colorectal cancer, pancreatic carcinoma, ovary cancer, stomach cancer, brain tumors, glioblastoma, astrocytoma, kidney cell carcinoma, non-Hodgkin lymphomas, uterus cancer, cervical cancer, liver cell carcinoma, gall bladder carcinoma, liver cancer, multiple myeloma, tumors of the throat and mouth, larynx cancer, bladder cancer, mesotheliomas, melanomas, multiple melanoma, esophageal cancer, thyroid cancer, testicular cancer, soft tissue sarcomas, leukemias, and fatigue during or after cancer treatment.

19. Use of alpha-GPC, or of a derivative thereof, or of a composition containing alpha-GPC and/or one or more derivatives thereof, for preparing a medicament for the treatment of cancers and tumor cachexia, and for supporting the convalescence after cancer or cancer treatment in humans.

20. A process for the treatment of cancers and tumor cachexia, and for supporting the convalescence after cancer or cancer treatment in humans, comprising administering alpha-GPC or a derivative thereof, or a composition containing alpha-GPC and/or one or more derivatives thereof, to a patient in need of such a treatment.
